(19) United States
(12) Patent Application Publication
(10) Pub. No.: US 2022/0061756 A1
(43) Pub. Date: Mar. 3, 2022

(54) HYDRATION ALARM DEVICES

(71) Applicant: Duke Energy Corporation, Charlotte, NC (US)

(72) Inventors: Shiva K Sainoju Korremla, Cary, NC (US); Steven Patrick Hinkel, California, KY (US); Danny Wayne Marrs, Conway, SC (US)

(21) Appl. No.: 17/412,334

(22) Filed: Aug. 26, 2021

Related U.S. Application Data
(60) Provisional application No. 63/070,958, filed on Aug. 27, 2020.

(51) Int. Cl. A61B 5/00; G01W 1/06

(52) U.S. Cl. CPC A61B 5/4875; G01W 1/06; A61B 5/6802

(57) ABSTRACT

Portable electronic devices are provided. A portable electronic device includes a weather sensor and a digital display. Moreover, the portable electronic device includes a vibrating motor and/or a buzzer. The digital display is configured to display a target level of liquids to be consumed by a user of the portable electronic device, in response to detection by the weather sensor of a weather condition.

### CROSS-REFERENCE TO RELATED APPLICATION

The present application claims priority to U.S. Provisional Patent Application No. 63/070,958, filed on Aug. 27, 2020, the entire content of which is incorporated herein by reference.

### FIELD

The present disclosure relates to portable electronic alarm devices that respond to weather conditions.

### BACKGROUND

Workers in harsh environments, such as utility field crews, may need to consume significant quantities of liquids (e.g., water) while working outdoors, especially during summer months. Though written hydration guidelines have been developed to inform workers in such crews about when and how much to hydrate, such guidelines are not personalized for each individual worker. Nor do such guidelines actively remind workers on an ongoing basis of a need to hydrate. Likewise, written cold-weather guidelines, which may be intended to protect workers from hyperthermia, frost bite, and the like, are not personalized for individual workers and do not actively remind workers on an ongoing basis of the need to take breaks during extreme cold weather.

### SUMMARY

A portable electronic device, according to some embodiments, may include a weather sensor, a digital display, a vibrating motor, and a buzzer. The vibrating motor and the buzzer may be configured to vibrate the portable electronic device and emit a sound, respectively, in response to detection by the weather sensor of a weather condition. Moreover, the digital display may be configured to display a target level of liquids to be consumed by a user of the portable electronic device, in response to the detection by the weather sensor of the weather condition.

In some embodiments, the weather sensor may include an ambient temperature sensor, an ambient light sensor, or a humidity sensor.

According to some embodiments, the digital display may have a pixel height of no more than sixty-four pixels. Moreover, the digital display may be further configured to display a time duration of a hydration break to be taken by the user, in response to the detection by the weather sensor of the weather condition.

In some embodiments, the portable electronic device may be a wearable device. Moreover, the portable electronic device may include a magnetic clip.

A portable electronic device, according to some embodiments, may include a weather sensor, a digital display, and a buzzer. The buzzer may be configured to emit a sound, in response to detection by the weather sensor of a weather condition. Moreover, the digital display may be configured to display a target level of liquids to be consumed by a user of the portable electronic device, in response to the detection by the weather sensor of the weather condition.

In some embodiments, the digital display may be further configured to display a time duration of a hydration break to be taken by the user, in response to the detection by the weather sensor of the weather condition.

According to some embodiments, the portable electronic device may be a wearable device. Moreover, the portable electronic device may include a magnetic clip.

A portable electronic device, according to some embodiments, may include a weather sensor, a digital display, and a vibrating motor. The vibrating motor may be configured to vibrate the portable electronic device, in response to detection by the weather sensor of a weather condition. Moreover, the digital display may be configured to display a target level of liquids to be consumed by a user of the portable electronic device and a recommended time duration of a hydration break to be taken by the user, in response to the detection by the weather sensor of the weather condition.

In some embodiments, the portable electronic device may include an expansion port. For example, the expansion port may include a lightning sensor. Moreover, the portable electronic device may be a wearable device and/or may include a magnetic clip.

### BRIEF DESCRIPTION OF THE DRAWINGS

FIG. 1A is a schematic illustration of a work area that includes portable electronic devices according to the present invention.

FIG. 1B is a block diagram of one of the portable electronic devices of FIG. 1A.

FIG. 1C is a block diagram that illustrates details of an example processor and memory that may be used in the portable electronic devices of FIG. 1A.

FIG. 2 is a flowchart of operations performed by the portable electronic devices of FIG. 1A.

### DETAILED DESCRIPTION

A portable electronic device according to the present invention can actively measure weather conditions that an individual worker is experiencing and can emit a haptic, audible, and/or visual alarm to the worker of a need for a hydration break. For example, the device may constantly measure ambient temperature, ambient light, and/or humidity. As a result, the device can reduce worker injuries and downtime due to heat-related (or cold-related) injuries. Moreover, in response to the measured weather conditions, the device can indicate a level (e.g., in liquid ounces per hour) of hydration needed. A single crew may include, as an example, twelve to sixteen workers. Because different workers in the same crew may experience different weather conditions (e.g., by working in the shade versus the sun), different devices worn by the different workers may indicate different, personalized levels of hydration and/or different hydration-break durations.

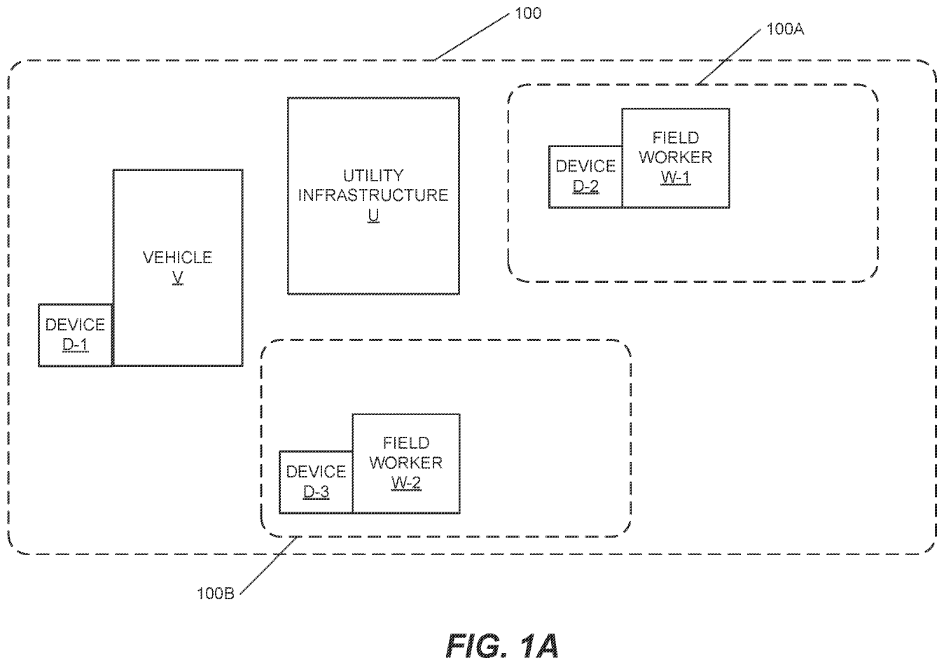

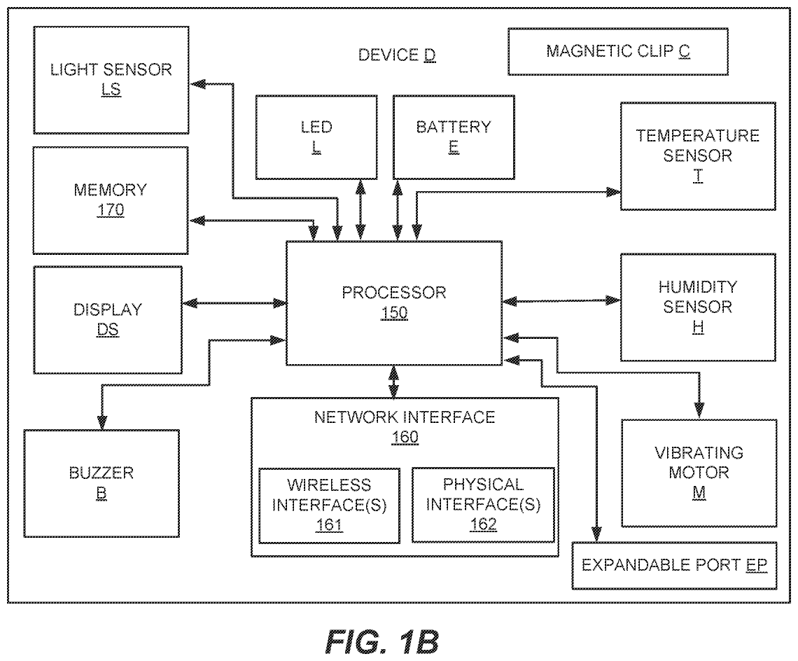

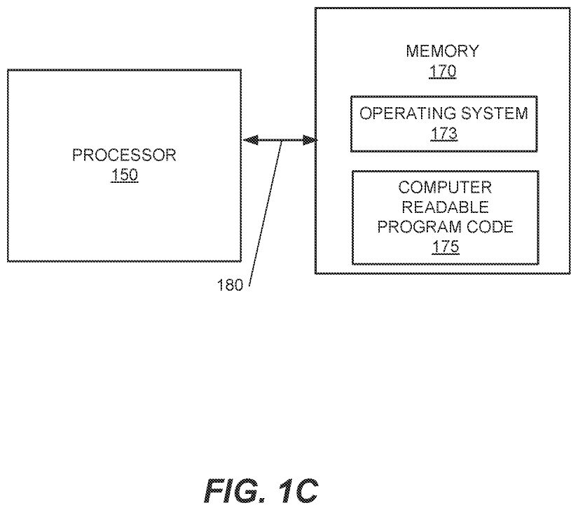

FIG. 1A is a schematic illustration of a work area 100 that includes outdoor environment such as utility infrastructure U. The infrastructure U may include any part of a network (e.g., a transmission/distribution network) that is operated by a utility, including, for example, one or more poles, one or more pipes, one or more cables, one or more wires, and/or one or more conduits. A plurality of field workers W may be working in the area 100. The area 100 may thus be referred to as the “field.”

Each worker W may wear (or otherwise keep nearby) a portable electronic device D that is configured to provide a hydration alarm, which indicates a need to hydrate. The worker W may thus also be referred to as a “user” of the device D. In some embodiments, each device D may include a magnetic clip C (FIG. 1B), which allows the device D to (a) clip onto a belt (or other clothing) of a worker W or to (b) magnetically attach to a metal portion (e.g., a door, hood, or tailgate) of a vehicle V, such as a utility truck. For example, a first worker W-1 may wear a device D-2, a second worker W-2 may wear a device D-3, and/or the vehicle V may have a device D-1 magnetically attached thereto.

The workers W-1 and W-2 may be in different portions 100A and 100B, respectively, of the area 100. For example, the portion 100A may have more shade (e.g., due to a nearby tree or structure) than the portion 100B, which may have more exposure to the sun. As a result, an ambient temperature may be lower for the worker W-1 than for the worker W-2.

FIG. 1B is a block diagram of a portable electronic device D of FIG. 1A. The device D may include a processor 150, a network interface 160, and a memory 170. The processor 150 of the device D may be coupled to the network interface 160. The processor 150 may be configured to communicate with other electronic devices (e.g., a smartphone, a tablet computer, a laptop computer, and/or a desktop computer) via the network interface 160.

For example, the network interface 160 may include one or more wireless interfaces 161 and/or one or more physical interfaces 162. The wireless interface(s) 161 may comprise wireless communications circuitry, such as BLUETOOTH® circuitry, cellular communications circuitry that provides a cellular wireless interface (e.g., 4G/5G/LTE, other cellular), and/or Wi-Fi circuitry. The physical interface(s) 162 may comprise wired communications circuitry, such as wired Ethernet, serial, and/or USB circuitry. In some embodiments, however, the wireless interface(s) 161 and/or the physical interface(s) 162 may be omitted from (or blocked from usage by) the device D, as utility company policy may prohibit network communications in certain circumstances.

The device D also includes one or more weather sensors that can detect a weather condition (e.g., exceeding or falling below a threshold ambient temperature) that triggers a need for hydration by a field worker W (FIG. 1A). The sensor(s) may include, for example, an ambient temperature sensor T, an ambient light (e.g., sunlight) sensor LS, and/or a humidity sensor H.

The device D further includes one or more alarm features. The alarm feature(s) may include, for example, a vibrating motor M, a buzzer B, and/or an LED L. As an example, the buzzer B may be a piezo buzzer. The motor M is configured to provide a vibration that can be felt by the worker W, the buzzer B is configured to emit a sound (e.g., a beep) that can be heard by the worker W, and the LED L is configured to provide a flashing light that can be seen by the worker W. Accordingly, in response to detection by the sensor(s) of a weather condition that corresponds to the need for hydration, the alarm feature(s) are configured to provide a haptic, audible, and/or visual alarm to the worker W.

Moreover, the device D may include a digital display DS. As an example, the display DS may be thirty-two or sixty-four pixels in height. The display DS can display information related to a hydration alarm. For example, as a supplement/complement to a haptic, audible, and/or visual alarm, the display DS can inform the worker W of a recommendation for a particular hydration target (e.g., 8-16, 12-20, 16-24, or 24-32 ounces of liquid per hour) and a recommendation for a particular time duration/length (e.g., 10, 15, or 20 minutes) of a hydration break.

In some embodiments, the device D may also include a battery E, which can be configured to supply power (e.g., via various respective electrical connections) to any other component that is shown in FIG. 1B. For simplicity of illustration, however, the battery E is only shown as being electrically connected to the processor 150. The battery E may be, for example, a lithium-ion rechargeable battery. Moreover, a group battery charger may, in some embodiments, be kept in a vehicle V (FIG. 1A) and may be configured to simultaneously recharge respective batteries E of a plurality of the devices D.

The device D may have a compact size. For example, the display DS may have a height of no more than sixty-four pixels, and overall dimensions of the device D may be no larger than five inches in length (e.g., in the height direction of the display DS), two inches in width, and one inch in depth. The device D may also be capable of expansion to perform further functions. As an example, the device D may include an expandable (or “expansion”) port EP, which may comprise a lightning sensor (e.g., a lightning detector) or another type of sensor.

The device D may, in some embodiments, also include an on/off switch, which may silence the buzzer B (and/or the motor M) but may not necessarily power off the display DS or the LED L. Moreover, the switch (and/or a different switch) may operate as a reset switch that resets the device D such that the device D will wait, for example, at least one hour (or some other predetermined time period) before triggering a subsequent hydration alarm. In some embodiments, however, a new hydration alarm (e.g., accompanied by the display of updated hydration target/duration information on the display DS) may automatically be triggered once per hour.

FIG. 1C is a block diagram that illustrates details of an example processor 150 and memory 170 that may be used in accordance with various embodiments. The processor 150 communicates with the memory 170 via an address/data bus 180. The processor 150 may be, for example, a commercially available or custom microprocessor. Moreover, the processor 150 may include multiple processors. The memory 170 may be a non-transitory computer readable storage medium and may be representative of the overall hierarchy of memory devices containing the software and data used to implement various functions of a portable electronic device D (FIGS. 1A and 1B) as described herein. The memory 170 may include, but is not limited to, the following types of devices: cache, ROM, PROM, EPROM, EEPROM, flash, static RAM (“SRAM”), and dynamic RAM (“DRAM”).

As shown in FIG. 1C, the memory 170 may hold various categories of software and data, such as computer readable program code 175 and/or an operating system 173. The operating system 173 controls operations of the device D. In some embodiments, the operating system 173 may manage the resources of the device D and may coordinate execution of various programs by the processor 150. For example, the computer readable program code 175, when executed by a processor 150 of the device D, may cause the processor 150 to perform any of the weather measurement and/or hydration alarm operations described herein.

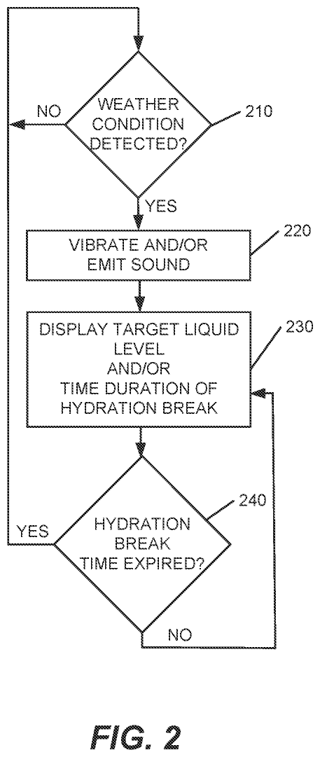

FIG. 2 is a flowchart of operations that can be performed by each portable electronic device D (FIGS. 1A and 1B). As shown in FIG. 2, a device D can detect (Block 210) a weather condition by using one or more weather sensors of the device D. In response to the detection, the device D can vibrate and/or emit a sound (Block 220) by using a vibrating motor M (FIG. 1B) and a buzzer B (FIG. 1B), respectively, of the device D. The device D can further respond to the detection by displaying (Block 230), via a display DS (FIG. 1B) of the device D, a target level of liquids to be consumed by a user of the device D. In some embodiments, the display DS can also display a time duration of a hydration break to be taken by the user, in response to the detection. For example, the display DS can continue to display the target level of liquids and/or the time duration of the hydration break until (Block 240) the time duration expires.

The present invention has been described above with reference to the accompanying drawings. The present invention is not limited to the illustrated embodiments. Rather, these embodiments are intended to fully and completely disclose the present invention to those skilled in this art. In the drawings, like numbers refer to like elements throughout. Thicknesses and dimensions of some components may be exaggerated for clarity.

Spatially relative terms, such as “under,” “below,” “lower,” “over,” “upper,” “top,” “bottom,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the example term “under” can encompass both an orientation of over and under. The device may be otherwise oriented (rotated 90 degrees or at other orientations) and the spatially relative descriptors used herein interpreted accordingly.

Herein, the terms “attached,” “connected,” “interconnected,” “contacting,” “mounted,” and the like can mean either direct or indirect attachment or contact between elements, unless stated otherwise.

Well-known functions or constructions may not be described in detail for brevity and/or clarity. As used herein the expression “and/or” includes any and all combinations of one or more of the associated listed items.

The terminology used herein is for the purpose of describing particular embodiments only and is not intended to be limiting of the present invention. As used herein, the singular forms “a,” “an,” and “the” are intended to include the plural forms as well, unless the context clearly indicates otherwise. It will be further understood that the terms “comprises,” “comprising,” “includes,” and/or “including” when used in this specification, specify the presence of stated features, operations, elements, and/or components, but do not preclude the presence or addition of one or more other features, operations, elements, components, and/or groups thereof

It will also be understood that although the terms “first” and “second” may be used herein to describe various elements, these elements should not be limited by these terms. These terms are only used to distinguish one element from another element. Thus, a first element could be termed a second element, and similarly, a second element may be termed a first element without departing from the teachings of present invention.

Example embodiments of the present invention may be embodied as devices and methods. Accordingly, example embodiments of present invention may be embodied in hardware and/or in software (including firmware, resident software, micro-code, etc.). Furthermore, example embodiments of present invention may take the form of a computer program product comprising a non-transitory computer-usable or computer-readable storage medium having computer-usable or computer-readable program code embodied in the medium for use by or in connection with an instruction execution system. In the context of this document, a computer-usable or computer-readable medium may be any medium that can contain, store, communicate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, or device. More specific examples (a nonexhaustive list) of the computer-readable medium would include the following: an electrical connection having one or more wires, a portable computer diskette, a random access memory (“RAM”), a read-only memory (“ROM”), an erasable programmable read-only memory (“EPROM” or Flash memory), an optical fiber, and a portable compact disc read-only memory (“CD-ROM”). The computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Example embodiments of present invention are described herein with reference to flowchart and/or block diagram illustrations. It will be understood that each block of the flowchart and/or block diagram illustrations, and combinations of blocks in the flowchart and/or block diagram illustrations, may be implemented by computer program instructions and/or hardware operations. These computer program instructions may be provided to a processor of a general purpose computer, a special purpose computer, or other programmable data processing apparatus to produce a machine, such that the instructions, which execute via the processor of the computer or other programmable data processing apparatus, create/use circuits for implementing the functions specified in the flowchart and/or block diagram block or blocks.

These computer program instructions may also be stored in a computer usable or computer-readable memory that may direct a computer or other programmable data processing apparatus to function in a particular manner, such that the instructions stored in the computer usable or computer-readable memory produce an article of manufacture including instructions that implement the functions specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart and/or block diagram block or blocks.

What is claimed is:

1. A portable electronic device comprising:
a weather sensor;
a digital display;
a vibrating motor; and
a buzzer,
wherein the vibrating motor and the buzzer are configured to vibrate the portable electronic device and emit a sound, respectively, in response to detection by the weather sensor of a weather condition, and
wherein the digital display is configured to display a target level of liquids to be consumed by a user of the portable electronic device, in response to the detection by the weather sensor of the weather condition.

2. The portable electronic device of claim 1, wherein the weather sensor comprises an ambient temperature sensor, an ambient light sensor, or a humidity sensor.

3. The portable electronic device of claim 1, wherein the digital display has a pixel height of no more than sixty-four pixels.

4. The portable electronic device of claim 1, wherein the digital display is further configured to display a time duration of a hydration break to be taken by the user, in response to the detection by the weather sensor of the weather condition.

5. The portable electronic device of claim 1, wherein the portable electronic device is a wearable device.

6. The portable electronic device of claim 1, further comprising a magnetic clip.

7. A portable electronic device comprising:
a weather sensor;
a digital display;
a buzzer,
wherein the buzzer is configured to emit a sound, in response to detection by the weather sensor of a weather condition, and
wherein the digital display is configured to display a target level of liquids to be consumed by a user of the portable electronic device, in response to the detection by the weather sensor of the weather condition.

8. The portable electronic device of claim 7, wherein the digital display is further configured to display a time duration of a hydration break to be taken by the user, in response to the detection by the weather sensor of the weather condition.

9. The portable electronic device of claim 7, wherein the portable electronic device is a wearable device.

10. The portable electronic device of claim 7, further comprising a magnetic clip.

11. A portable electronic device comprising:
a weather sensor;
a digital display; and
a vibrating motor;
wherein the vibrating motor is configured to vibrate the portable electronic device, in response to detection by the weather sensor of a weather condition, and
wherein the digital display is configured to display a target level of liquids to be consumed by a user of the portable electronic device and a recommended time duration of a hydration break to be taken by the user, in response to the detection by the weather sensor of the weather condition.

12. The portable electronic device of claim 11, further comprising an expansion port.

13. The portable electronic device of claim 12, wherein the expansion port comprises a lightning sensor.

14. The portable electronic device of claim 11, wherein the portable electronic device is a wearable device.

15. The portable electronic device of claim 11, further comprising a magnetic clip.
